(19) United States
(12) Patent Application Publication
(10) Pub. No.: US 2025/0255539 A1
(43) Pub. Date: Aug. 14, 2025

(54) CERVICAL DILATION MONITORING SYSTEMS AND METHODS

(71) Applicant: CERV, INC., WILMINGTON, DE (US)

(72) Inventors: Elizabeth Buzbee, St. Louis, MO (US); Annika Avula, St. Louis, MO (US); Sandra Montgomery, St. Louis, MO (US)

(21) Appl. No.: 19/034,771

(22) Filed: Jan. 23, 2025

Related U.S. Application Data
(60) Provisional application No. 63/552,709, filed on Feb. 13, 2024.

(51) Int. Cl. A61B 5/00; A61B 1/00; A61B 1/04; A61B 1/06; A61B 1/303

(52) U.S. Cl. CPC A61B 5/435; A61B 1/00009; A61B 1/00011; A61B 1/00045; A61B 1/00089; A61B 1/04; A61B 1/0661; A61B 1/303; A61B 2562/0257

(57) ABSTRACT

Disclosed herein are novel cervical dilation monitoring systems and methods. In some embodiments, the systems and methods can include a novel cervical dilation monitoring device that collects continuous, objective, and reliable data regarding the dilation of a patient during labor. The device can include a cup that can be positioned within the vaginal canal of a patient. It can also include an imaging unit, a proximity sensor, and one or more light sources positioned within the cup that can be directed toward the patient's cervix. The imaging unit can capture visual data from the cervix or cervical os and the proximity sensor can capture data regarding a distance between the imaging unit and the cervix. The data can be transmitted to a processor or a computing device that can perform image analysis to convert the data into cervical dilation measurements.

### CROSS-REFERENCE TO RELATED APPLICATION

This application claims the benefit of priority from U.S. Provisional Patent Application No. 63/552,709, filed on Feb. 13, 2024, which is incorporated herein by reference in its entirety.

### TECHNICAL FIELD

The present disclosure relates to cervical dilation monitoring systems and methods.

### BACKGROUND

During labor, the cervix undergoes effacement (e.g., thinning) and dilation (e.g., widening) to facilitate delivery of the fetus. The dilation process takes place over several hours, and clinicians typically monitor the cervical os (i.e., the opening that forms as the cervix dilates) at regular intervals throughout the process. Cervical dilation provides critical information about the labor process to the clinician. However, the standard method for measuring cervical dilation-digital examination-is subjective and prone to error. To perform a digital examination, the clinician places two fingers into the cervical opening to estimate the cervical dilation based on the distance between their fingers. This method requires the clinician to estimate the diameter of the cervix based on subjective feel alone. Thus, digital examination does not provide objective and reliably accurate data. The digital method also only provides for data to be collected periodically, often with hours passing between measurements. However, because cervical dilation is not a linear process, non-continuous measurements can lead to inaccurate prediction of labor outcomes. In addition, digital examination can be very uncomfortable and invasive for many patients, and repeated digital examination may increase the risk of, for example, transmitting bacteria or other pathogens to the patient.

A rare alternative solution for measuring cervical dilation is transperineal ultrasound, which is primarily used for women at risk of preterm birth. However, this method has also been shown to produce error. More importantly, ultrasound technology is expensive, meaning it would be infeasible to use transperineal ultrasound in place of digital examination in a large number of cases.

Thus, there is a need for new or improved methods and systems for monitoring cervical dilation.

### SUMMARY

Disclosed herein are novel cervical dilation monitoring systems and methods. Some examples are or include systems or methods that can resolve the problems discussed herein or not mentioned in this disclosure but known to a person having ordinary skill in the art.

In some embodiments, the systems and methods can include a novel cervical dilation monitoring device that collects continuous, objective, and reliable data regarding the dilation of a patient during labor. The device can include a cup that can be positioned within the vaginal canal of a patient. It can also include an imaging unit, a proximity sensor, and one or more light sources positioned within the cup that can be directed toward the patient's cervix. The imaging unit can capture visual data from the cervix or cervical os and the proximity sensor can capture data regarding a distance between the imaging unit and the cervix. The data can be transmitted to a processor or a computing device that can perform image analysis to convert the data into cervical dilation measurements.

In some embodiments, the dilation monitoring device can include a cup positioned within the vaginal canal of the patient and an imaging unit, a proximity sensor, and one or more light sources disposed within the cup. The imaging unit, proximity sensor, and one or more light sources can each be oriented toward the patient's cervix or the cervical os. Thus, the imaging unit can be capable of collecting visual data of the cervix or the cervical os while the proximity sensor can be capable of collecting data regarding the distance between the imaging unit and the cervix. A processor or computing device can be in communication with the imaging unit and the proximity sensor and can be configured to receive data transmitted therefrom. The processor or computing device can process the data, or it can transmit the data to an external device for processing. Either the processor or the computing device can use image processing software to convert the raw data collected by the imaging unit and the proximity sensor into cervical dilation measurements which can then be provided to one or more clinicians providing care to the patient. The dilation monitoring device may only need to be inserted into the vaginal canal once and can then collect and transmit data continuously throughout the dilation process.

These and other important aspects of the invention are described more fully in the detailed description below. The invention is not limited to the particular assemblies, apparatuses, methods, and systems disclosed herein. Other embodiments can be used and changes to the described embodiments can be made without departing from the scope of the claims that follow the detailed description.

### BRIEF DESCRIPTION OF THE DRAWINGS

The present disclosure will be understood more fully from the detailed description given below and from the accompanying drawings of various example embodiments of the disclosure.

FIG. 1 illustrates a partial diagram of a female anatomy during the dilation process.

FIG. 2 illustrates a plan view of a dilation monitoring device constructed and positioned, in accordance with some embodiments of the present disclosure.

FIG. 3 illustrates a diagram of the dilation monitoring device of FIG. 2 configured to collect data regarding the dilation of a patient during labor.

FIG. 4 illustrates a block diagram of example aspects of an example computing system, in accordance with some embodiments of the present disclosure.

FIG. 5 illustrates an example method of the technologies disclosed herein, in accordance with some embodiments of the present disclosure.

### DETAILED DESCRIPTION

The present disclosure will be understood more fully from the detailed description given below and from the accompanying drawings of various example embodiments of the disclosure.

Disclosed herein are novel cervical dilation monitoring systems and methods. In some embodiments, the systems and methods can include a novel cervical dilation monitoring device that collects continuous, objective, and reliable data regarding the dilation of a patient during labor. The device can include a cup that can be positioned within the vaginal canal of a patient. It can also include an imaging unit, a proximity sensor, and one or more light sources positioned within the cup that can be directed toward the patient's cervix. The imaging unit can capture visual data from the cervix or cervical os and the proximity sensor can capture data regarding a distance between the imaging unit and the cervix. In some embodiments, the distance is based on converting the pixels of the captured dilation to a number in centimeters for the dilation. The data can be transmitted to a processor or computing device that can perform image analysis to convert the data into cervical dilation measurements.

In some embodiments, the dilation monitoring device can include a cup positioned within the vaginal canal of the patient and an imaging unit, a proximity sensor, and one or more light sources disposed within the cup. The imaging unit, proximity sensor, and one or more light sources can each be oriented toward the patient's cervix or the cervical os. Thus, the imaging unit can be capable of collecting visual data of the cervix or the cervical os while the proximity sensor can be capable of collecting data regarding the distance between the imaging unit and the cervix. A processor or computing device can be in communication with the imaging unit and the proximity sensor and can be configured to receive data transmitted therefrom. The processor or computing device can process the data, or it can transmit the data to an external device for processing.

Either the processor or computing device can use image processing software to convert the raw data collected by the imaging unit and the proximity sensor into cervical dilation measurements which can then be provided to one or more clinicians providing care to the patient. Also, aspects of effacement and fetal station can be captured and measured. In some examples, the device is further configured for measuring the effacement of a patient or measuring fetal position within the patient.

The image processing software can include machine learning, such as supervised or unsupervised learning. Also, the software can include an artificial neural network. In some embodiments, random forests can be used for classification, regression, and other tasks in training one or more models of the software. Further, the software can use any known computer vision computing techniques to enhance the measurements or the captured data.

The dilation monitoring device may only need to be inserted into the vaginal canal once and can then collect and transmit data continuously throughout the dilation process. And, in some embodiments, the device can be secured onto a hospital gown or hospital bed. In other embodiments, the device can be secured to a leg band.

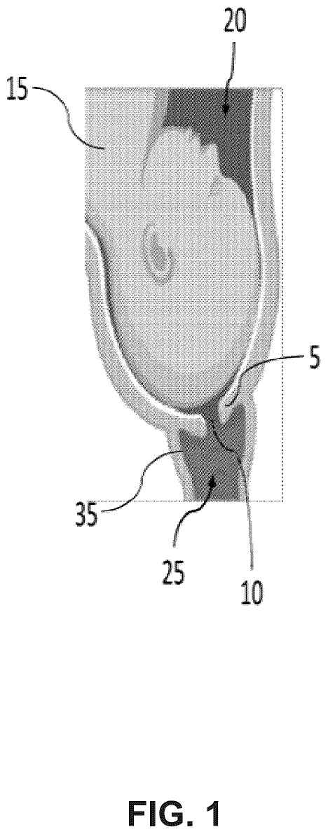

Referring to FIG. 1, a cervix 5 may become dilated during labor. During dilation, the cervix 5 may thin and widen and a cervical os 10 may form to allow a fetus 15 to pass from a uterus 20 to a vaginal canal 25. During the dilation process, monitoring the size of the cervical os 10 over time may provide clinicians with critical information about the patient or the progression of the labor process.

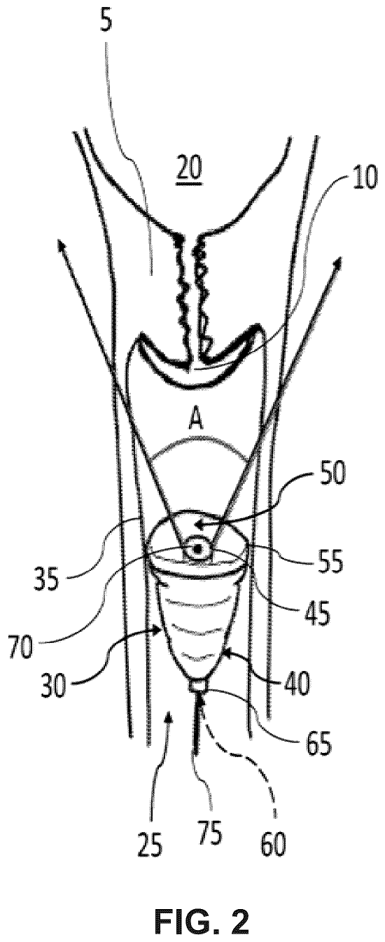

Referring to FIG. 2, a dilation monitoring device 30 can be positioned within the vaginal canal 25. For example, the dilation monitoring device 30 can be surrounded by a vaginal wall 35 of the vaginal canal 25. The dilation monitoring device 30 can include a cup 40 and an imaging unit 45 positioned within the cup 40 and configured to collect visual data from the cervix 5 or the cervical os 10. In some embodiments, the device can interface with the cervix. In other embodiments, the device does not interface with the cervix. In some instances, the cup 40 can include medical-grade silicone. In some instances, the cup 40 can include another type of material that is flexible and capable of conforming to the shape of the vaginal canal 25 (such as a material that is commonly used as an alternative to medical grade silicone). The cup 40 can be formed from a material that is hypoallergenic, non-reactive, or biocompatible.

The cup 40 can be configured to securely position the imaging unit 45 within the vaginal canal 25 and to stabilize the imaging unit 45 with respect to the cervical os 10 during use. For example, the cup 40 can be arranged such that at least a portion of the cup 40 can contact the vaginal wall 35 and generate suction, which can create at least a partial seal between the cup 40 and the vaginal wall 35. The at least partial seal between the cup 40 and the vaginal wall 35 can immobilize the cup 40 (and thus the imaging unit 45) with respect to the cervical os 10. With such stabilization, the cup 40 can improve the ability of the imaging unit 45 to collect consistent and accurate visual data from the cervix 5 or the cervical os 10. In some embodiments, the cup includes holes to allow for fluids to pass through without compromising the stability in the vaginal canal. In such examples, the cup can use friction for maintaining position instead of suction.

The cup 40 can include a first opening 50 disposed at a first end 55 of the cup 40 proximate to the cervix 5. A second opening 60 can be disposed at a second end 65 of the cup 40 opposing the first end 55. The first opening 50 can be larger than the second opening 60. The first opening 50 can provide a window through which the imaging unit 45 can gather data. For example, the imaging unit 45 can have a field of view that extends outwardly from the first opening 50 of the cup 40 and can allow the imaging unit 45 to view at least a portion of the cervix 5 and the cervical os 10. The field of view of the imaging unit 45 (i.e., the area within which the imaging unit 45 can capture data) can be defined by an angle A. For example, the angle A can be imparted with a value of 60 degrees. In other instances, the angle A can be imparted with a value of 45 degrees, 80 degrees, 90 degrees, or any other suitable value.

In some examples, the entirety of the cervix 5 is visible within the field of view of the imaging unit 45. And, the cup 40 can be positioned or constructed such that the imaging unit 45 is maintained about 2-4 centimeters away from the cervix 5, in which in some cases, the entirety of the cervix 5 is visible to the imaging unit 45.

The imaging unit 45 can include a camera that is suitable for use in a wet or dark environment (e.g., an endoscope). For example, the imaging unit 45 can be provided in the form of a medical-grade endoscope camera. In some instances, the imaging unit 45 can include another type of imaging device. The imaging unit 45 can include a lens 70 arranged to face in the direction of the cervical os 10. One or more light sources (not depicted) can be positioned proximate to the lens 70 and can direct light in the direction of the cervix 5 and the cervical os 10. For example, the one or more light sources can include light emitting diodes (LEDs) or any other suitable light producing device. The one or more light sources can illuminate the cervix 5 and or the cervical os 10 such that the imaging unit 45 can collect useful visual data regarding the dilation process. Visual data collected by the imaging unit 45 can be communicated to one or more external devices either via a wired connection or via a wireless connection (e.g., via Wi-Fi, Bluetooth, or any other suitable wireless communication protocol).

In some examples, the dilation monitoring device 30 can include a proximity sensor (not shown) disposed within the cup 40 and positioned proximate to the lens 70. The proximity sensor can be configured to measure a distance between the lens 70 and the cervical os 10. Also, the proximity sensor can be positioned at a distance from the cervical os 10 as the lens 70. The proximity sensor can include an infrared (IR) sensor, ultrasonic sensor, laser distance sensor, photoelectric sensor, or the like, or a combination thereof. The proximity sensor can be configured to communicate data to one or more external devices either via a wired connection or via a wireless connection (e.g., via Wi-Fi, Bluetooth, or any other suitable wireless communication protocol).

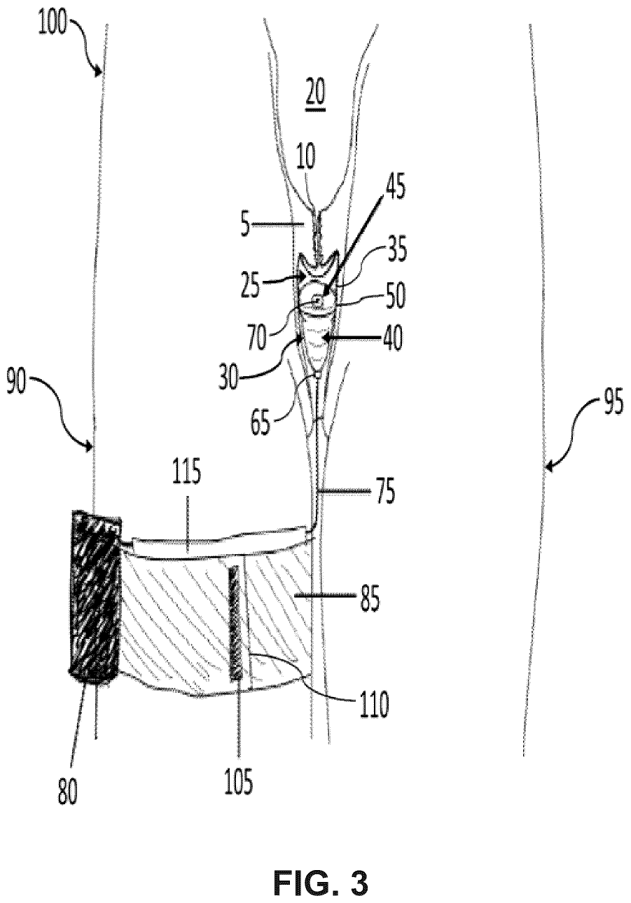

The second opening 60 can allow a wire 75 to extend into the cup 40. The wire 75 can supply power to the imaging unit 45. The wire 75 can also supply power to one or more of the light sources and the proximity sensor. Additionally, in some examples, the wire 75 can facilitate communication between the imaging unit 45, the proximity sensor, and one or more external devices. For example, as shown in FIG. 3, the wire 75 can be connected to a computing device 80 configured to receive, process, or store data collected by the imaging unit 45 or the proximity sensor.

The computing device 80 can be coupled to or positioned on a leg strap 85. The leg strap 85 can be provided in the form of a strip of stretchable material (e.g., elastic, spandex, elastane, lycra, neoprene, and the like) configured to wrap around either a right leg 90 or a left leg 95 of a patient 100. The leg strap 85 can include a closure 105 positioned proximate to an end region 110 of the leg strap 85. The closure 105 can facilitate coupling of the end region 110 of the leg strap 85 to an adjacent portion of the leg strap 85 in order to secure the leg strap 85 around one of the legs 90, 95 of the patient 100. For example, the closure 105 can include hook-and-loop fasteners (e.g., VELCRO). Alternatively, the closure 105 can include clasps, buttons, magnets, or other suitable fastening mechanisms. When the closure 105 is engaged, the position of the leg strap 85 along one of the legs 90, 95 of the patient 100 can be fixed, for example, due to the tension produced by the stretchability of the material of the leg strap 85.

In some instances, a wire casing 115 can be disposed along the wire 75. In the example of FIG. 3, the wire casing 115 encases a portion of the wire 75 and can be disposed along the wire 75 between the second end 65 of the cup 40 and the computing device 80. Alternatively, the wire casing 115 can encase a larger or smaller portion of the wire 75 than the portion of the wire 75 shown as encased within the wire casing 115 in FIG. 3. In yet other alternatives, the wire casing 115 can be omitted. The wire casing 115 can provide protection or insulation to the wire 75. The wire casing 115 can be provided for aesthetic purposes. In some instances, the wire casing 115 can be coupled to or formed integrally with the leg strap 85. Also, the wire casing 115 can restrict the movement of the wire 75. For example, the wire casing 115 can prevent the wire from being unintentionally pulled, which may cause a pulling of the dilation monitoring device 30 away from the cervix 5 or even unintentionally removing the dilation monitoring device 30 from the vaginal canal 25.

In some embodiments, the dilation monitoring device 30 can be used to monitor the cervix 5 or the cervical os 10 throughout the entirety of the dilation process. The dilation monitoring device 30, once inserted, can perform continuous data collection, whereas prior methods (e.g., digital examination) require repeated insertion and only allow periodic data collection.

Data collected by the imaging unit 45 and the proximity sensor can be transmitted to the computing device 80 via the wire 75 or via wireless communication. The device 80 can, in turn, transmit the data collected by the imaging unit 45 and the proximity sensor to a computer (not depicted in FIG. 2 or 3). The computer can include one or more software which can allow the computer to perform image analysis on the data received. For example, the one or more software programs can include an artificial intelligence algorithm designed to perform image recognition or image analysis. In this way, the computer can use the data transmitted by the imaging unit 45 and the proximity sensor to calculate a measurement of the dilation of the cervix 5 at any given point in time. The computer can further include one or more software programs designed to translate the measurements calculated by the computer into one or more graphical displays which can communicate the measurements to one or more clinicians (such as via a display). For example, the computer can use the data it receives to generate a graphical representation of the dilation of the patient 100 over time.

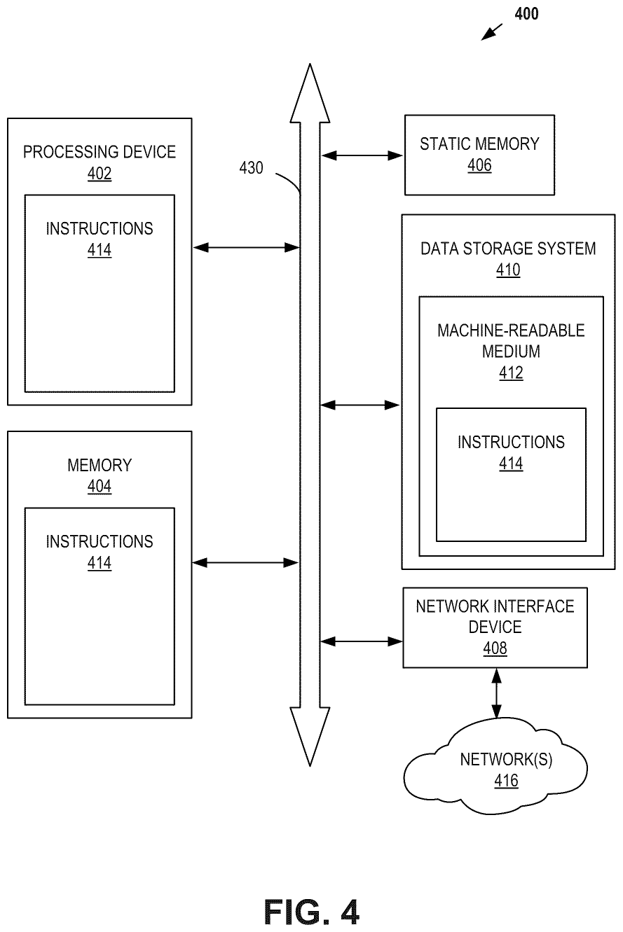

FIG. 4 illustrates a block diagram of example aspects of an example computing system 400 that can include, be a part of, or be connected to the computing device 80, in accordance with some embodiments of the present disclosure. FIG. 2 illustrates parts of the computing system 400 within which a set of instructions for causing a machine of the computing system 400 to perform any one or more of the methodologies discussed herein can be executed. In some embodiments, the computing system 400 can correspond to a host system that includes, is coupled to, or utilizes memory or can be used to perform the operations of a controller. In alternative embodiments, the machine can be connected (e.g., networked) to other machines in a LAN, an intranet, an extranet, or the Internet. The machine can operate in the capacity of a server or a client machine in client-server network environment, as a peer machine in a peer-to-peer (or distributed) network environment, or as a server or a client machine in a cloud computing infrastructure or environment. The machine can be a personal computer (PC), a tablet PC, a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computing system 400 can include a computing device that includes a processor 402, a main memory 44 (e.g., read-only memory (ROM), flash memory, dynamic random-access memory (DRAM), etc.), a static memory 406 (e.g., flash memory, static random-access memory (SRAM), etc.), and a data storage system 410, which communicate with each other via a bus 430. The processor 402 represents one or more general-purpose processing devices such as a microprocessor, a central processing unit, or the like. More particularly, the processing device can be a microprocessor or a processor implementing other instruction sets, or processors implementing a combination of instruction sets. The processor 402 can also be one or more special-purpose processing devices such as an application specific integrated circuit (ASIC), a field programmable gate array (FPGA), a digital signal processor (DSP), network processor, or the like. The processing device 202 is configured to execute instructions 414 for performing the operations discussed herein. The computing system 400 can further include a network interface device 408 to communicate over one or more LAN/WAN networks 416. The data storage system 410 can include a machine-readable storage medium 212 (also known as a computer-readable medium) on which is stored one or more sets of instructions 414 or software embodying any one or more of the methodologies or functions disclosed herein. The instructions 414 can also reside, completely or at least partially, within the main memory 404 or within the processor 402 during execution thereof by the computing system 400, the main memory 404 and the processor 402 also constituting machine-readable storage media.

With respect to some embodiments, disclosed herein are systems and methods that can include computerized methods for monitoring cervical dilation of a patient, as well as include a non-transitory computer-readable storage medium for carrying out technical operations of the computerized methods. The non-transitory computer-readable storage medium has tangibly stored thereon, or tangibly encoded thereon, computer-readable instructions that when executed by one or more devices (e.g., one or more personal computers or servers) cause at least one processor to perform a method for monitoring cervical dilation of a patient.

With respect to some embodiments, a system is provided that includes at least one computing device configured to monitor cervical dilation of a patient via data captured by the monitoring device. And, with respect to some embodiments, a corresponding method is provided to be performed by at least one computing device and the monitoring device. In some example embodiments, computer program code can be executed by at least one processor of one or more computing devices to implement functionality in accordance with at least part of some embodiments disclosed herein; and the computer program code being at least a part of or stored in a non-transitory computer-readable medium.

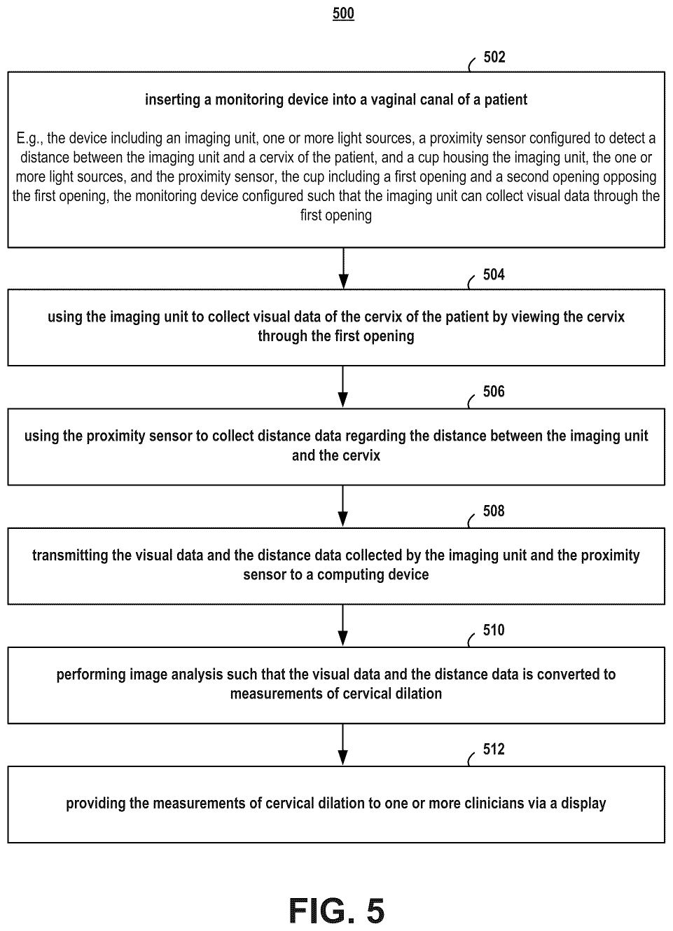

For example, FIG. 5 shows a method 500 for monitoring cervical dilation of a patient via a computing device and a monitoring device. In some examples, the method 500 can be combined with any one or more of the methods, systems, apparatuses, or devices disclosed herein. As shown in FIG. 5, the method 500 starts with, at step 502, inserting a monitoring device into the vaginal canal of a patient. In some embodiments, the monitoring device includes an imaging unit, one or more light sources, a proximity sensor configured to detect a distance between the imaging unit and a cervix of the patient, and a cup housing the imaging unit, the one or more light sources, and the proximity sensor. The cup can include a first opening and a second opening opposing the first opening, and the monitoring device can be configured such that the imaging unit can collect visual data through the first opening.

The method 500 also includes, at step 504, using the imaging unit to collect visual data of the cervix of the patient by viewing the cervix through the first opening. Also, the method 500 includes, at step 506, using the proximity sensor to collect distance data regarding the distance between the imaging unit and the cervix. Then, at step 508, the method continues with transmitting the visual data and the distance data collected by the imaging unit and the proximity sensor to a computing device. And, at step 510, the method includes performing image analysis such that the visual data and the distance data is converted to measurements of cervical dilation. At step 512, the method 500 then continues with providing the measurements of cervical dilation to one or more clinicians via a display. In some embodiments, the distance is based on converting the pixels of the captured dilation to a number in centimeters for the dilation.

In the foregoing specification, embodiments of the disclosure have been described with reference to specific example embodiments thereof. It will be evident that various modifications can be made thereto without departing from the broader spirit and scope of embodiments of the disclosure as set forth in the following claims. The specification and drawings are, accordingly, to be regarded in an illustrative sense rather than a restrictive sense.

What is claimed is:

1. A system, comprising:
a cup, comprising:
a first end, and a second end opposing the first end; and
a first opening positioned at the first end, and a second opening positioned at the second end; and
an imaging unit within the cup, the imaging unit configured to capture visual data through the first opening to measure cervical dilation of a patient.

2. The system of claim 1, further a computing device communicably coupled to the imaging unit configured to process the data to measure cervical dilation of the patient.

3. The system of claim 1, further comprising a light source configured to direct light through the first opening.

4. The system of claim 1, further comprising a proximity sensor within the cup, wherein the sensor is configured to capture a distance between the imaging unit and a cervix of the patient.

5. The system of claim 4, further comprising a light source configured to direct light through the first opening, and wherein the cervix is illuminated by the light source.

6. The system of claim 5, wherein the cup, the imaging unit, and the proximity sensor are configured to be inserted together into a vaginal canal of the patient and oriented such that the imaging unit captures visual data of the cervix, which is illuminated by the light source.

7. The system of claim 1, wherein the cup and imaging unit are part of an intravaginal monitoring device for measuring cervical dilation of a patient.

8. The system of claim 7, wherein the device is further configured for measuring effacement of a patient.

9. The system of claim 7, wherein the device is further configured for measuring fetal position within the patient.

10. A method for monitoring cervical dilation of a patient, comprising:
inserting a monitoring device into a vaginal canal of a patient, the monitoring device including an imaging unit, one or more light sources, a proximity sensor configured to detect a distance between the imaging unit and a cervix of the patient, and a cup housing the imaging unit, the one or more light sources, and the proximity sensor, the cup including a first opening and a second opening opposing the first opening, the monitoring device configured such that the imaging unit can collect visual data through the first opening;
using the imaging unit to collect visual data of the cervix of the patient by viewing the cervix through the first opening;
using the proximity sensor to collect distance data regarding the distance between the imaging unit and the cervix;
transmitting the visual data and the distance data collected by the imaging unit and the proximity sensor to a computing device;
performing image analysis such that the visual data and the distance data is converted to measurements of cervical dilation; and
providing the measurements of cervical dilation to one or more clinicians via a display.

11. A system, comprising:
a cup, comprising:
a first end, and a second end opposing the first end; and
a first opening positioned at the first end, and a second opening positioned at the second end;
an imaging unit within the cup, the imaging unit configured to capture visual data through the first opening to measure cervical dilation of a patient; and
a computing device communicably coupled to the imaging unit configured to process the data to measure cervical dilation of a patient.

12. The system of claim 11, further comprising a light source configured to direct light through the first opening.

13. The system of claim 12, further comprising a proximity sensor within the cup.

14. The system of claim 13, wherein the sensor is positioned adjacent to the imaging unit.

15. The system of claim 13, wherein the cup, the imaging unit, and the proximity sensor are configured to be inserted together into a vaginal canal of the patient and oriented such that the imaging unit captures visual data of a cervix of the patient, which is illuminated by the light source.

16. The system of claim 15, wherein the proximity sensor is configured to capture a distance between the imaging unit and the cervix.

17. The system of claim 16, wherein the distance is determined is measured and identified by the computing device.

18. The system of claim 11, further comprising a proximity sensor within the cup.

19. The system of claim 18, wherein the sensor is positioned adjacent to the imaging unit.

20. The system of claim 18, wherein the cup, the imaging unit, and the proximity sensor are configured to be inserted together into a vaginal canal of the patient and oriented such that the imaging unit captures visual data of a cervix of the patient.
